(19) United States
(12) Patent Application Publication
(10) Pub. No.: US 2009/0098110 A1
(43) Pub. Date: Apr. 16, 2009

(54) INJECTABLE FORMS OF SOLID-FORMING CROSSLINKED BIOELASTIC BIOPOLYMERS FOR LOCAL DRUG DELIVERY

(75) Inventors: Samuel B. Adams, Jr., Durham, NC (US); Lori A. Setton, Durham, NC (US)

Correspondence Address: MYERS BIGEL SIBLEY & SAJOVEC, PO BOX 37428, RALEIGH, NC, 27627, US

(73) Assignee: Duke University, Durham, NC (US)

(21) Appl. No.: 12/129,085

(22) Filed: May 29, 2008

Related U.S. Application Data
(60) Provisional application No. 60/940,813, filed on May 30, 2007.

(51) Int. Cl. A61K 39/395; A61K 38/02

(52) U.S. Cl. 424/130.1; 514/2

(57) ABSTRACT

A method for delivering a compound of interest to a selected region in a subject is carried out by administering a composition to the region of interest, the composition comprising a cross-linked bioelastomer in combination with the compound of interest non-covalently combined with the cross-linked bioelastomer. In some embodiments, the composition is produced by the process of cross-linking a bioelastomer to produce a cross-linked polymer thereof, and then combining said cross-linked polymer with said compound of interest. Methods of making the compositions, and kits useful for carrying out the method, are also described.

### RELATED APPLICATIONS

This application claims the benefit of U.S. patent application Ser. No. 60/940,813, filed May 30, 2007, the disclosure of which is incorporated by reference herein in its entirety.

### GOVERNMENT FUNDING

This invention was made with Government support under grant numbers EB002263 and AR052745 from the National Institutes of Health. The Government has certain rights to this invention.

### FIELD OF THE INVENTION

The present invention concerns methods and compositions for the controlled released delivery of pharmaceutical compounds.

### BACKGROUND OF THE INVENTION

Infections associated with open fractures and implanted prosthetic devices can present a clinical and economical challenge. It has been reported that about 100,000 infections related to fracture fixation devices and approximately 12,000 infections related to primary implantation of joint prostheses occur each year in the United States; thus totaling $15,000 and $30,000, respectively, per infection for medical treatment. Therefore, local antibiotic delivery systems are becoming increasingly popular in orthopaedic surgery for the treatment and prophylaxis of musculoskeletal infections. Such systems can potentially provide the advantages of site-specific delivery, ease of administration, improved patient compliance over long-term oral or IV administration, and decreased overall drug dosing with concurrent decreased side effects associated with systemic administration.

The concept of local antibiotic delivery systems for orthopaedic surgery was first introduced in 1970, by Buchholz through the use of gentamicin impregnated polymethylmethacrylate (PMMA) for the treatment prosthetic joint infections. Since this report, PMMA has become the gold standard biomaterial for local antibiotic delivery to orthopaedic infections. However, intrinsic to the properties that make PMMA a good candidate for bone cement lie disadvantages for drug delivery. PMMA requires reaction temperatures up to 100° C., most of the loaded drug remains trapped, it must be administered trough an open surgical procedure, and it is not biodegradable, requiring a secondary surgery for removal. A biodegradable local antibiotic delivery system would serve the orthopaedic surgery community very well.

Many candidate molecules have been identified as potential drugs for the treatment of OA, for their ability to modify the progression of disease. Some molecules include the protein drugs, TNF-a inhibitors and IL-1 inhibitors, that have shown significant promise both in preclinical and clinical studies in preventing the onset and retarding the progression of the disease.

In the treatment of orthopaedic infections, localized delivery of the antibiotic or other therapeutic is highly preferred. One such technique is the administration of the drug directly into the affected joint cavity, i.e. intra-articular injection, which is currently recommended for corticosteroids and hyaluronan solutions in treating OA. Although the intra-articular mechanism of drug delivery is attractive to the patient and clinician alike, it is compromised by the presence of a highly efficient lymphatic system that rapidly clears molecules from the synovial cavity. Consequently, the therapeutic drug has to be administered frequently or at high concentrations to be effective. This, in turn, may be costly and result in adverse side effects and high levels of patient discomfort. Controlled drug delivery systems have been sought-after to overcome these challenges.

U.S. Pat. No. 6,328,996 to Urry describes bioelastomeric drug delivery systems, but does not suggest them for administration directly into a region of interest.

### SUMMARY OF THE INVENTION

The present invention provides a method for delivering a compound of interest to a selected region in a subject, said method comprising: administering a composition to said region of interest, said composition comprising a cross-linked bioelastomer in combination with said compound of interest non-covalently combined with said cross-linked bioelastomer. In some embodiments, the composition is produced by the process of cross-linking a bioelastomer to produce a cross-linked polymer thereof, and then combining said cross-linked polymer with said compound of interest. In some particular embodiments, the composition is produced by the process of cross-linking a bioelastomer (e.g., with a cross-linking agent such as THPP) to produce a cross-linked polymer thereof, drying said cross-linked polymer, rehydrating said cross-linked polymer in the presence of a quenching agent (e.g., glycine) in an aqueous solution in an amount sufficient to inactivate remaining active sites on said cross-linked polymer and again drying said cross-linked and quenched polymer, then combining said cross-linked and quenched polymer with said compound of interest, in solution, to produce said composition with said compound of interest non-covalently combined with in said cross-linked polymer.

In some embodiments (e.g., where the compound of interest is an antibiotic, antifungal, or other non-oligomeric organic compound), the composition comprises said compound of interest and said cross-linked polymer at a molar ratio of at least 0.5 to 1, drug to polymer.

In some embodiments, such as where the compound of interest is a protein or peptide, the composition comprises the compound of interest and the cross-linked polymer at a molar ratio of at least 0.05 to 1, or even 0.1 to 1, drug to polymer.

A further aspect of the invention is a method of making a composition useful for delivering a compound of interest to a selected region in a subject, said method comprising: cross-linking a bioelastomer to produce a cross-linked polymer thereof, and then combining said cross-linked bioelastomer with said compound of interest to produce said composition, with said compound of interest non-covalently combined with said cross-linked polymer.

A still further aspect of the invention is a kit useful for making a composition for delivering a compound of interest to a selected region in a subject, said kit comprising:

a first reagent, said first reagent produced by the process of cross-linking an elastin-like peptide to produce a cross-linked bioelastomeric polymer thereof (and wherein said cross-linked polymer is preferably quenched to inactivate remaining reactive sites thereon), and a second reagent comprising said compound of interest non-covalently entrapped in said cross-linked polymer.

Preferably, the resulting crosslinked bioelastomer exhibits the a phase transitioning behavior, as does the uncrosslinked native bioelastomer. Preferably, the phase transitioning behavior of crosslinked bioelastomers as used herein is reversible and repeatable. Preferably, the compositions herein have a viscosity suitable for injection or delivery through a syringe.

A further aspect of the invention is an injection device (including syringes and other injection devices) containing a composition as described herein.

A further aspect of the invention is the use of an injection device as described herein for carrying out a method as described herein.

In some embodiments the composition comprises a bioelastomer or modified bioelastomer that undergoes a visually observable transition at a temperature below the temperature of the selected region in the subject. That is, this injectable liquid does transition to a gel (a consistency more viscous than the liquid) above a specified temperature. The temperature at which this transition occurs may be any suitable temperature, for example a temperature between from 20 or 3 degrees C. up to 35 or 40 degrees C.

### BRIEF DESCRIPTION OF THE DRAWINGS

FIG. 1: Average percentage of starting vancomycin released from compound-loaded 150 mg/ml crosslinked bioelastic polymer constructs.

FIG. 2: Average percentage of starting vancomycin released from compound-loaded 225 mg/ml crosslinked bioelastic polymer constructs.

FIG. 3: Average percentage of starting cefazolin released from compound-loaded 150 mg/ml crosslinked bioelastic polymer constructs.

FIG. 4: Average percentage of starting cefazolin released from compound-loaded 225 mg/ml crosslinked bioelastic polymer constructs.

FIG. 5: An agar plate showing diameters of zone of inhibition (dark circles) of vancomycin released from a crosslinked bioelastic polymer construct and serial vancomycin dilutions (underlined values). As shown, values for day 1 constructs correspond to ˜1 mg/ml of active compound (day 7 values correspond to less than 0.1 mg/ml active drug).

FIG. 6: An agar plate showing diameters of zone of inhibition of cefazolin released from a crosslinked bioelastic polymer construct and serial cefazolin dilutions (underlined values).

FIG. 7: Viscosity comparison of uncross-linked bioelastic polymer at 150 mg/ml to the crosslinked bioelastic polymer described herein at 150 mg/ml

### DETAILED DESCRIPTION OF THE PREFERRED EMBODIMENTS

“Bioelastic polymer” as used herein refers to compounds that comprise repeating elastomeric units. The elastomeric units are typically pentapeptides, tetrapeptides, or nonapeptides. Examples are elastin-like polypeptides or “ELPs”. “Crosslinked bioelastic polymers” as used herein refers to bioelastic polymers that have undergone a treatment (e.g., mixing with chemical crosslinking agent, gamma irradiation, light or UV exposure, mixing with enzymatic initiator) that promotes the formation of covalent, inter-molecular and intra-molecular bonds between amino acid residues of the bioelastic polymer. A unique feature of the crosslinking process is that the crosslinked bioelastic polymers retain the properties of an injectable solution and form a gel upon undergoing a thermally-induced phase transition.

Subjects” as used herein includes human subjects (including both male and female subjects and including juvenile, adolescent, adult, and geriatric subjects), as well as animal subjects, particularly other mammalian subjects such as primates, dogs, cats, and horses, for veterinary purposes.

“Arthritis” as used herein means any type of arthritis, including but not limited to rheumatoid arthritis, osteoarthritis, septic arthritis (bacterial or fungal) and ankylosing spondylitis.

“Osteomyelitis” as used herein means any type of infection of the bone of acute or chronic duration.

“Aggregate” as used herein means a collection or agglomeration of a plurality of molecules such that a particle is formed.

“Dis-aggregation” as used herein means a separation of molecule or molecules from an aggregate, a progressive diminishing of aggregate size; or a progressive diminishing of particle size.

“Region of interest” as used herein may be any region of interest, including but not limited to joints. In some embodiments of joint administration, the region of interest is an intervetebral disk space or a related spinal joint structure.

“Body temperature” as used herein includes both core body temperature and regional body temperature (e.g., the temperature of an extremity when a region of interest such as a joint is located in an extremity).

“Joint” as used herein refers to a movable point where two bones meet, often a synovial or diarthrodial joint or syndesmosis, and including but not limited to ball and socket joints, ellipsoidal joints, gliding joints, hinge joints, pivot joints and saddle joints. Such joints may be located in, for example, the shoulder, neck, spine, elbow, hip, wrist, hand, knee, ankle, or foot

“Injection” or “injecting” as used herein may be carried out by any suitable means through a needle, syringe, shunt, cannula (e.g., of 7-33 gauge) or any other suitable device that delivers the composition to be delivered directly into the region of interest (in contrast to and not including systemic delivery), as a single bolus or as an infusion over time.

“Antibody” or “antibodies” as used herein refers to all types of immunoglobulins, including IgG, IgM, IgA, IgD, and IgE. The term “immunoglobulin” includes the subtypes of these immunoglobulins, such as IgG₁, IgG₂, IgG₃, IgG₄, etc. Of these immunoglobulins, IgM and IgG are preferred, and IgG is particularly preferred. The antibodies may be of any species of origin, including (for example) mouse, rat, rabbit, horse, or human, or may be chimeric antibodies. The term “antibody” as used herein includes antibody fragments which retain the capability of binding to a target antigen, for example, Fab, F(ab′)₂, and Fv fragments, and the corresponding fragments obtained from antibodies other than IgG. Such fragments are also produced by known techniques.

The disclosures of all United States patent references cited herein are to be incorporated by reference herein in their entirety.

Compounds of interest. Any suitable compound, including but not limited to proteins and peptides, may be mixed with the cross-linked polymer as described herein. In general, the compound of interest (or combination of two or more compounds of interest) is a therapeutic agent(s).

For example, the compound of interest may be selected from the group of bone morphogenetic proteins, peptides, and growth factors. See, e.g., U.S. Pat. No. 6,593,394.

The compound of interest may be selected from the group of antiinfectives such as antibiotics and antiviral agents; anti-rejection agents; analgesics and analgesic combinations; anti-inflammatory agents (i.e., anti-interleukins or anti-TNF agents); hormones such as steroids; growth factors, including bone morphogenic proteins (i.e. BMP's 1-7), bone morphogenic-like proteins (i.e. GDF-5, GDF-7 and GDF-8), epidermal growth factor (EGF), fibroblast growth factor (i.e. FGF 1-9), platelet derived growth factor (PDGF), insulin like growth factor (IGF-I and IGF-II), transforming growth factors (i.e. TGF-.beta. I-III), vascular endothelial growth factor (VEGF); and other naturally derived or genetically engineered proteins, polysaccharides, glycoproteins, or lipoproteins. See, e.g., D. Overaker, U.S. Pat. No. 6,575,986.

The compound of interest may be compounds or agents that actually promote or expedite healing, the effectors may also include compounds or agents that prevent infection (e.g., antimicrobial agents and antibiotics), compounds or agents that reduce inflammation (e.g., anti-inflammatory agents), compounds that prevent or minimize adhesion formation, such as oxidized regenerated cellulose (e.g., INTERCEED™ and SURGICEL™, available from Ethicon, Inc.), hyaluronic acid, and compounds or agents that suppress the immune system (e.g., immunosuppressants). Suitable compounds of interest include heterologous or autologous growth factors, proteins (including matrix proteins), peptides, antibodies, enzymes, platelets, glycoproteins, hormones, cytokines, glycosaminoglycans, nucleic acids, analgesics, viruses, virus particles, and cell types. It is understood that one or more effectors of the same or different functionality may be incorporated within the crosslinked bioelastic polymer. Suitable compounds of interest include the multitude of heterologous or autologous growth factors known to promote healing and/or regeneration of injured or damaged tissue. Suitable compounds of interest include chemotactic agents; therapeutic agents (e.g., antibiotics, steroidal and non-steroidal analgesics and anti-inflammatories, anti-rejection agents such as immunosuppressants and anti-cancer drugs); various proteins (e.g., short term peptides, bone morphogenic proteins, glycoprotein and lipoprotein); cell attachment mediators; biologically active ligands; integrin binding sequence; ligands; various growth and/or differentiation agents and fragments thereof (e.g., epidermal growth factor (EGF), hepatocyte growth factor (HGF), IGF-I, IGF-II, TGF-.beta. I-III, growth and differentiation factors, vascular endothelial growth factors (VEGF), fibroblast growth factors (FGF), platelet derived growth factors (PDGF), insulin derived growth factor (IGF) and transforming growth factors, parathyroid hormone, parathyroid hormone related peptide, bFGF; TGFB superfamily factors; BMP-2; BMP-4; BMP-6; BMP-12; sonic hedgehog; GDF5; GDF6; GDF8; MP52, CDMP1); small molecules that affect the upregulation of specific growth factors; tenascin-C; hyaluronic acid; chondroitin sulfate; fibronectin; decorin; thromboelastin; thrombin-derived peptides; heparin-binding domains; heparin; heparan sulfate; DNA fragments and DNA plasmids. Suitable effectors likewise include the agonists and antagonists of the agents noted above. The growth factor can also include combinations of the growth factors listed above. In addition, the growth factor can be autologous growth factor that is supplied by platelets in the blood, connective tissue in the bone marrow or other tissue source. In this case, the growth factor from platelets will be an undefined cocktail of various growth factors. See, e.g., J. Hwang et al., US Patent Application Publication No. 2004/0267362.

In some embodiments, particularly for the treatment of arthritis, the compound of interest is an antiinflammatory agent, examples of which include but are not limited to TNF blocking antibodies, IL-1 antibodies, soluble TNF receptors, soluble IL-1 receptors, IL-1 receptor antagonists, COX-2 inhibitors, and non-steroidal antiinflammatory agents (including active fragments thereof for protein or peptide compounds such as antibodies and receptors). Numerous examples of such compounds are known. See, e.g., U.S. Pat. No. 6,846,834; see also U.S. Pat. Nos. 6,624,184; 6,596,746; 6,498,165; and 6,420,373. One particular example is rhIL1Ra (including its isoforms)(commercially available as KINERET™ from Amgen), as described in U.S. Pat. Nos. 6,599,873 and 5,075,222.

In some particular embodiments, such as for the treatment of arthritis (including rheumatoid arthritis and psoriatic arthritis), plaque psoriasis, Crohn's disease, ulcerative colitis, and ankylosing spondylitis, the compound of interest is an antibody that specifically binds to human tumor necrosis factor-alpha (TNF alpha). See, e.g., U.S. Pat. Nos. 6,835,823; 6,790,444; 6,652,863; 6,284,471; 6,277,969; 5,919,452; 5,698,195; 5,656,272; 5,641,751; and 5,223,395 One example of such antibodies that can be used to carry out the present invention is infliximab (commercially available from Centocor as REMICAD® brand infliximab).

In some particular embodiments, such as for the treatment of osteoporosis, the compound of interest is a therapeutic bisphosphonate, examples of which include but are not limited to etidronate, clodronate, pamidronate, alendronate, (6-amino-1-hydroxyhexylindene)bis-phosphonate, tiludronate, risedronate, (3-(dimethylamino)-1-hydroxypropylidene)bis-phosphonate, (1-hydroxy-3(methylpentylamino)propylidene)bis-phosphonate (BM 21.0955), (1-hydroxy-3-(1-pyrrolidinyl)propylidene)bis-phosphonate (EB-1053) zoledronic acid, olpadronic acid, incadronic acid, NE-10244, YH-529 and mixtures thereof. See, e.g., U.S. Pat. No. 5,735,810.

Antibiotics that can be used to carry out the present invention include but are not limited to glycopeptides (such as vancomycin and teicoplanin), aminoglycosides (such as gentamicin and tobramycin), cephalosporins (such as cefazolin, cefuroxime, cefotaxime, cefipeme), fluoroquinolones (such as ciprofloxacin or levofloxacin), tetracyclines (such as doxycycline or tetracycline), macrolides (such as erythromycin or clarithromycin), penicillins (such as amoxicillin or ampicillin), sulfonamides, carbapenems, isoniazid, linezolid, metronidazole, clindamycin, polymyxin, rifampin, bacitracin, neomycin, chloramphenical, oxolinic acid, nalidix acid, pefloxacin, piracil, etc., including combinations thereof.

Antifungals that can be used to carry out the present invention include but are not limited to polyenes (such as nystatin or amphotericin B), imadazoles (such as miconazole or ketoconazole), triazoles (such as fluconazole or itraconazole), allylamines (such as terbinifine or butenafine), echinocandins, griseofulvin, etc., including combinations thereof.

Bioelastic polymers. Bioelastic polymers are known and described in, for example, U.S. Pat. No. 5,520,672 to Urry et al. In general, bioelastic polymers are polypeptides comprising elastomeric units of bioelastic pentapeptides, tetrapeptides, and/or nonapeptides. Thus in some embodiments the elastomeric unit is a pentapeptide, in other embodiments the elastomeric unit is a tetrapeptide, and in still other embodiments the elastomeric unit is a nonapeptide. Bioelastic polymers that may be used to carry out the present invention are set forth in U.S. Pat. No. 4,474,851, which describes a number of tetrapeptide and pentapeptide repeating units that can be used to form a bioelastic polymer. Specific bioelastic polymers that can be used to carry out the present invention are also described in U.S. Pat. Nos. 4,132,746; 4,187,852; 4,500,700; 4,589,882; and 4,870,055. Still other examples of bioelastic polymers are set forth in U.S. Pat. No. 6,699,294 to Urry, U.S. Pat. No. 6,753,311 to Fertala and Ko, U.S. Pat. No. 6,063,061 to Wallace and U.S. Pat. No. 6,852,834 to Chilkoti. The bioelastic polymers may contain additional residues or units such as leader and/or trailer sequences as is known in the art.

In one nonlimiting example, the bioelastic polymers used to carry out the present invention are polypeptides that contain at least the general block structure, [(VPGXⁱG)ₙⁱ]ₘ (SEQ ID NO:4) for i=1 to J, Xⁱ is any amino acid other than proline (e.g., Ala, Leu, Phe), nⁱ is the number of successive repeats of block i adjacent to and preceding block i+1. nⁱ is any number such as 1, 2, 3, or 4 up to 20 and nⁱ are independent of i. J is any number such as 1, 2, or 3 to 5 or more. m is the number of repeats of the block structure where m is any suitable number such as 2, 3 or 4 up to 60, 80 or 300 or more. The identity and frequency of the various amino acids at the fourth position can be changed. For example, the bioelastic polymers used to carry out the present invention may be polypeptides of the general formula: [(VPGAG)₅¹(VPGVG)²(VPGGG)₂³]ₘ, (SEQ ID NO:5) where X¹=A, X²=V, X³=G, n¹ is 5, n² is 1, n³ is 2, m is at least 1, 2, or 3 up to 100, 150 or 300 or more.

In another nonlimiting example, bioelastic polymers used to carry out the present invention may comprise repeating elastomeric units selected from the group consisting of bioelastic nonapeptides, pentapeptides and tetrapeptides, where the repeating units comprise amino acid residues selected from the group consisting of all amino acid and glycine residues. Preferred amino acid residues are selected from the group consisting of alanine, valine, leucine, isoleucine, proline, phenylalanine, tryptophan, and methionine. In many cases, the first amino acid residue of the repeating unit is a residue of valine, leucine, isoleucine or phenylalanine; the second amino acid residue is a residue of proline; the third amino acid residue is a residue of glycine; and the fourth amino acid residue is glycine or a very hydrophobic residue such as tryptophan, phenylalanine or tyrosine. Particular examples include the tetrapeptide Val-Pro-Gly-Gly (SEQ ID NO:6), the tetrapeptide GGVP (SEQ ID NO:7), the tetrapeptide GGFP (SEQ ID NO:8), the tetrapeptide GGAP (SEQ ID NO:9), the pentapeptide is Val-Pro-Gly-Val-Gly (SEQ ID NO:10), the pentapeptide GVGVP (SEQ ID NO:11), the pentapeptide GKGVP (SEQ ID NO:12), the pentapeptide GVGFP (SEQ ID NO:13), the pentapeptide GFGFP (SEQ ID NO:14), the pentapeptide GEGVP (SEQ ID NO:15), the pentapeptide GFGVP (SEQ ID NO:16), and the pentapeptide GVGIP (SEQ ID NO:17). See, e.g., U.S. Pat. No. 6,699,294 to Urry.

Cross-linking of bioelastic polymers. Cross-linking of the bioelastic polymers can be carried out in accordance with known techniques (see, e.g., U.S. Pat. No. 6,328,996 to Urry) or variations thereof that will be apparent to those skilled in the art. Examples of crosslinking techniques include, but are not limited to, preparation of the bioelastic polymer with glutaraldehyde, formaldehyde, chemical crosslinking agents, naturally-derived crosslinking agents (e.g., transglutaminase, genipin), or exposure of the bioelastic polymer to UV, gamma or other high energy sources that promote crosslinking (See, e.g., Lee J, et al., Biomacromolecules. 2(1): 170-9 (2001); Urry D W, et al., Biopolymers; 25 Suppl: S209-28 (1986); Urry D W et al., Biochemistr, 15(18): 4083-9 (1976); Nowatzki P J, Tirrell D A. Biomaterials. 25(7-8): 1261-7 (2004); Welsh E R, Tirrell D A, Biomacromolecules, 1(1): 23-30 (2000); Lim D W, et al., Biomacromolecules, 8(5): 1463-70 (2007); Trabbic-Carlson, K.; Setton, L. A.; Chilkoti, A. Biomacromolecules 4, 572-580 (2003); McHale, M. K.; Setton, L. A.; Chilkoti, A. Tissue Engineering 11, 1768-1779 (2005)). Thus, any suitable cross-linking agent can be used. Crosslinking of the bioelastic polymer is preferably performed such that the physical properties (e.g., viscosity) of the crosslinked bioelastic polymer permit injection of the bioelastic polymer to a local region.

In some embodiments, cross-linking agents used to carry out the present invention are preferably amine-free compounds containing hydroxymethyl phosphines (>P—CH₂—OH) that react with primary and secondary amines, generating stable aminomethylphosphines (>P—CH₂—N<) and only water as a byproduct of the crosslinking reaction via Mannich type condensation reaction. The crosslinking agents are amine-free compounds containing hydroxyalkyl, preferably hydroxymethyl, phosphine (HMP) groups, as described in U.S. Pat. No. 5,948,386 to Katti et al. In one embodiment, such compounds have the basic formula, hydroxymethyl phosphines (HMP): R—P(CH₂OH)₂, where R is any suitable organic molecule or group, such as —CH₂OH, —H, —C₂H₅, —C₆H₅, —COOH, —COOCH₃, —COOC₂H₅, etc. For example, tris(hydroxymethylphosphine), P(CH₂OH)₃ (or “THP”) is the basic crosslinker for this basic category. In a second embodiment, such compounds have the complex formula that contains at least one or more of the basic formula, hydroxymethylphosphines (HMP): R—P(CH₂OH)₂, where R is any suitable organic molecule or group, such as —CH₂OH, —H, —C₂H₅, —C₆H₅, —COOH, —COOCH₃, —COOC₂H₅, etc. For example, this group includes combined molecules of the two basic hydroxymethyl phosphines, 1,2-bis(bis(hydroxymethyl)phosphino)-benzene (HMPB), 1,2-bis(bis(hydroxymethyl)phosphino) ethane (HMPE) that are converted from mono and multiprimary phosphines, 1,2-bis(phosphino)-benzene, 1,2-bis(phosphino)ethane (PE). In a third embodiment, such compounds include homo- and hetero-functional crosslinking agents where the molecules having the hydroxymethylphosphine group are attached to the same as the hydroxymethyl phosphine containing molecules and to the other chemically active groups different from the hydroxymethyl phosphine groups. Thus particular examples of suitable cross-linking agents include tris(hydroxymethyl)phosphine (THP) and B-(tris-(hydroxymethyl)phosphino) propionic acid (THPP). The cross-linking may occur in sequential steps of adding additional cross-linker before achieving the desired cross-linking density.

Further preparation of cross-linked bioelastic polymers. After cross-linking as described above, the crosslinked bioelastic polymers may then be combined with a compound of interest, in an appropriate molar ratio to produce the desired compound loading. For example, in some embodiments (e.g., antibiotics) the composition comprises or consists essentially of the compound of interest and the cross-linked polymer at a molar ratio of at least 0.5 to 1, 1 to 1; or in some embodiments 2:1, drug to polymer, and up to 5 to 1 or more. In other embodiments (e.g., where the compound of interest is a protein or peptide, such as an antibody), the composition comprises or consists essentially of the compound of interest and the cross-linked polymer at a molar ratio of at least 0.05 to 1, 0.1 to 1, or 0.2 to 1, up to 1 to 1 or more, drug to polymer.

In some embodiments, the cross-linked bioelastic polymers are optionally dried (e.g., freeze-dried or lyophilized) and rehydrated in an aqueous solution (preferably in a volume of the aqueous solution equal to or less than that of the water volume lose during the drying step), prior to the step of combining the same with the compound of interest.

In some embodiments, the cross-linked bioelastic polymers are quenched following the crosslinking step, but prior to the combining step to inactivate remaining reactive sites, or cross-linking sites, to which the active agent might otherwise bind. Quenching can be carried out by adding a quenching agent such as glycine to the cross-linked bioelastic polymers at a suitable time in the preparation (e.g., following cross-linking but before drying; following cross-linking and before combining with the compound of interest without an intervening drying step; after drying and during rehydrating; etc.). The glycine solution is added to the crosslinked bioelastic polymer in an aqueous solution (preferably in a volume of aqueous solution equal to or less than that of the initial water volume fraction, or that water volume fraction lost during the dehydration step). The resulting composition may be purified by any suitable means, such as washing, dialysis or chromatography, to remove undesired residual ingredients. Additional desired ingredients such as preservatives, stabilizers, buffers or the like can be added at any appropriate point during the preparation of the composition. The resulting composition may be further dried as a means for storing the composition for future rehydration in aqueous solution with or without addition of preservatives, stabilizers, buffers or the like, prior to administration.

Administration. Patients afflicted with any disease or condition for which a depot drug is desired can be treated by these methods, with a particular example being patients afflicted with single joint infection, or single joint arthrides such as arthritis, osteoarthritis, in need of a joint replacement such as a knee or hip joint replacement, etc.

As noted above, a method of the invention generally comprises administering a composition to region of interest (e.g., directly, such as by injection in or to the region of interest). Alternatively, the composition can be swabbed, brushed, or applied directly to a region by any suitable means, such as to bone or tissue adjacent a joint in a patient undergoing a joint replacement (e.g., a knee or hip joint replacement) or other joint surgery. Examples of regions of interest include sites of bacterial or fungal infection which include but are not limited to bones (osteomyelitis) or the joint space (septic arthritis), muscles, or potential spaces created by accumulating bacteria between fascial planes.

The compound of interest (e.g., the compound of interest or drug) can be administered in any suitable amount depending upon the site of injection, age, weight, and condition of the subject, particular active agent, etc. In general the compound is administered in an amount of from 0.001, 0.01, 0.1, 1, 5 or 10 milligrams per administration or injection, up to about 100, 200 or 300 milligrams per administration or injection. In general the compound is administered at a concentration of 1 pg/ml to 500 mg/ml, with the injection volume dependent upon the region of interest. In general the crosslinked bioelastic polymer may also be co-administered at an equivalent concentration of 1 pg/ml to 500 mg/ml, or much higher concentrations in the example of mixing compound with elastomer. In some embodiments the compound of interest preferably has an in vivo half life in said region of one or two hours or more; in some embodiments administration of the compound of interest in combination with crosslinked bioelastic polymer in the said administration method is associated with an in vivo half life for the compound in said region of at least ½, 1 or 2 days, one week, or two weeks or more, up to 2 or three months or more. Stated otherwise, it is contemplated that the administering step will be carried out on a regular and repeated basis until resolution of the acute condition (e.g., for the treatment of an orthopaedic infection) or chronic condition (e.g., for the treatment of a chronic condition such as arthritis) defined by the release characteristics of the compound in question, thereby retaining the desired effective levels of the compound at the site of interest. The present invention is explained in greater detail in the following non-limiting Examples.

### EXAMPLE 1

### Bioelastomer Expression and Purification and Crosslinked Bioelastomer Preparation

Genetically-engineered elastin-like polypeptides (ELPs) are one class of bioelastic polymer that consist of multiple repeats of the pentapeptide VPGXG where X is any amino acid except for proline. ELPs of the notation ELP[KV₁₆-102] (MW=42.7 kDa, denoting incorporation of K and V in the guest residue position at a frequency of 1:16), were synthesized using a genetic engineering method, where the monomer of the ELP gene was chemically synthesized and oligomerized using recursive directional ligation (RDL) (See, e.g., D E Meyer and A Chilkoti, Synthesis of artificial polypeptides of specified molecular weight and sequence by recursive directional ligation of a synthetic gene: Examples from the elastin-like polypeptide system, Biomacromolecules 2: 357-367 (2002); see also A. Bandiera et al., Expression and characterization of human-elastin-repeat-based temperature-responsive protein polymers for biotechnological purposes, Biotechnol. Appl. Biochem. 42: 247-256 (2005)). The protein was expressed in Escherichia coli BLR(DE3) and purified from cell lysates using inverse transition cycling (ITC). The purified ELPs were concentrated to 150 mg/ml and 225 mg/ml as measured by UV-Vis spectrophotometry and stored at −80° C. until ready for use.

A trifunctional, water-soluble, amine-reactive cross-linker, β-[Tris(hydroxymethyl)phosphino]proprionic acid (THPP) (Pierce Biotechnology, Rockford, Ill.) was solubilized in 200 μl of phosphate buffered saline to a final concentration of 250 mg/ml. Aliquots of this solution were stored at −80° C. until ready for use.

Three microliters of the THPP solution were added to 400 μl of the KV₁₆-102 solution at a concentration of 150 mg/ml via manual pipetting. The resultant mixture was then agitated for 30 minutes at 4° C. to provide for thorough mixing. Subsequently, the mixture was brought to 37° C. and the cross-linking reaction was carried out for 24 hrs. Next, the mixture was brought to 4° C., frozen at −80° C. for 30 minutes, and lyophilized for 24 hrs. The resultant lyophilized structure was rehydrated for 24 hrs with a solution containing 4:1 molar equivalents of glycine molecules to the total number of reactive primary amines on the THPP molecules, prepared in an aqueous solution of volume corresponding to 90% of the water weight lost during lyophilization step #1 (based on 1 mg/ml water density). This was to ensure THPP binding site quenching in order to inhibit covalent bonding of the compound to reactive amines on the THPP. The rehydrated mixture was again brought to 4° C., frozen at −80° C. for 30 minutes, and lyophilized for 24 hrs (lyophilization step #2).

### EXAMPLE 2

### Compound Loading into the Crosslinked Bioelastomer Preparation

Thirty five of the previously mentioned constructs were made. Five constructs were rehydrated with a PBS solution of volume corresponding to 90% of the weight of water lost during the lyophilization step #2. Five constructs each were rehydrated with 5 mg, 10 mg, or 20 mg of vancomycin hydrochloride (MW=1485.74 Da) (Sigma-Aldrich, Inc, St. Louis, Mo.) dissolved in a PBS solution of volume corresponding to 90% of the weight of water lost during the lyophilization step #2. Five constructs each were rehydrated with 5 mg, 10 mg, or 20 mg of cefazolin sodium salt (MW=476.49 Da) (Sigma-Aldrich, Inc, St. Louis, Mo.) dissolved in a PBS solution of volume corresponding to 90% of the weight of water lost during the lyophilization step #2. All constructs were rehydrated for 24 hrs at 4° C. under gentle agitation.

To test the effect of ELP concentration on drug release, 15 additional samples were created, following the methods mentioned above, except that the concentration of ELP used was 225 mg/ml. Five of these constructs were rehydrated in PBS only, five were rehydrated in a PBS solution containing 7.5 mg of vancomycin hydrochloride, and five were rehydrated in a PBS solution containing 7.5 mg of cefazolin sodium salt. These drug amounts were intentionally chosen to keep the same molar ratio for the 225 mg/ml samples to that of the 150 mg/ml samples loaded with five milligrams of each antibiotic for comparison.

### EXAMPLE 3

### Antibiotic Release from the Crosslinked Bioelastomer Constructs

After compound (antibiotic) loading, 3 ml of 37° C. PBS were added to each sample (volume much greater than the water volume lost during either lyophilization step) and the samples were placed at 37° C. The samples underwent a phase transition (translucent to opaque) nearly instantaneously when placed at 37° C. This phase transitioning behavior was demonstrated to be reversible and repeatable. For the vancomycin samples, the volume of the supernatant was completely removed and replaced by fresh PBS on days 1-14, 21, and 28, in order to retain dilute compound concentrations throughout the experiment; these are the required conditions for the case of predicting kinetics of unsteady, one-dimensional solute transport. For the cefazolin samples, the supernatant was similarly removed and completely replaced on days 1-10. Antibiotic concentrations were measured in the supernatant using a UV/Vis spectrophotometer. Prior to measurement, a standard curve for each antibiotic was created using concentrations ranging from 15 mg/ml to 1.0 μg/ml for vancomycin (R²=1.0000) and 15 mg/ml to 0.1 μg/ml for cefazolin (R²=0.9999). Vancomycin concentrations were determined by taking the absorbance value at 280 nm in each sample and subtracting the average absorbance value of the five ELP control samples for a given timepoint. The same method was performed for cefazolin using the absorbance value at 270 nm. The remaining supernatant was stored at −80° C. until further use. These processes were the same for both the 150 mg/ml and 225 mg/ml constructs. After the final day of testing, the samples were brought to 4° C. and resuspended in 2 ml of PBS and mixed uniformly with manual pipetting.

### EXAMPLE 4

### Determining Biological Activity of the Antibiotics Released from the Crosslinked Bioelastomer Constructs

To determine if the eluted antibiotics remained active, bioassays were performed in triplicate, for a subset of samples, using Bacillus subtilis (ATCC 6633) for both vancomycin and cefazolin. Vancomycin supernatants from days 1, 7, 14, 21, and 28, as well as a sample of the final construct (following resolubilization) were tested against vancomycin serial dilutions of 5 mg/ml, 1 mg/ml, 0.5 mg/ml, 0.1 mg/ml, and 0.05 mg/ml. Cefazolin supernatants from days 1-3, as well as a sample of the final homogenized construct were tested against cefazolin serial dilutions of 2 mg/ml, 1 mg/ml, and 0.5 mg/ml. Bacterial suspensions were prepared in tryptic soy broth following the recommendations set forth by the manufacturer for Bacillus subtilis (Lyfo Disk, MicroBiologics, St. Cloud, Minn.). After 30 minutes of incubation at 37° C. the suspensions were swabbed onto petri dishes containing Mueller-Hinton agar. Twenty microliters of each supernatant were pipetted onto 6 mm filter paper disks applied to the surface of the agar. The plates were incubated for 18 hours at 37° C. After incubation, zones of inhibition were measured twice in two randomly chosen directions. Supernatants from ELP control samples were tested in this same manner.

### EXAMPLE 5

### Results: Vancomycin Release from the Crosslinked Bioelastomer Constructs

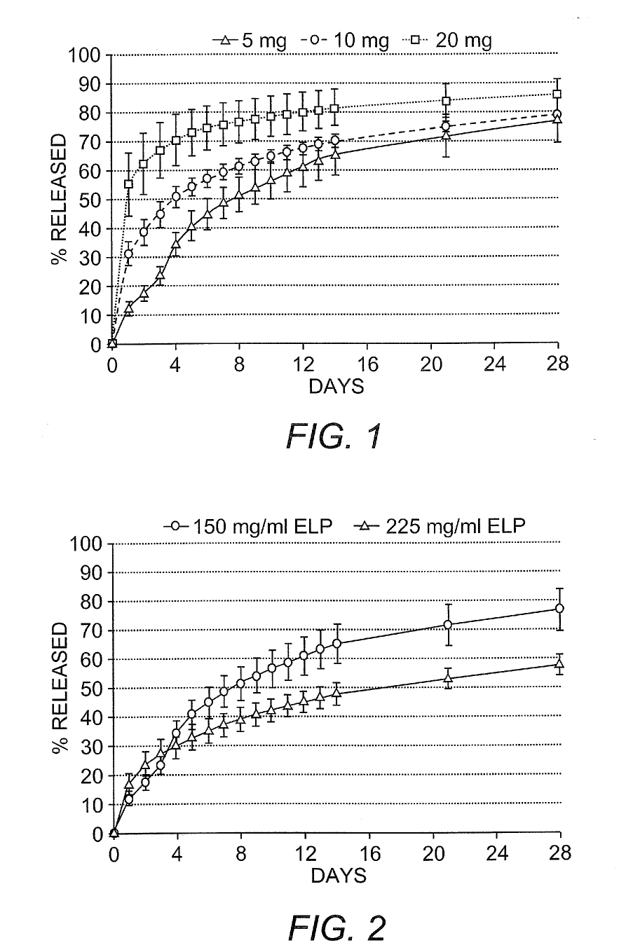

FIG. 1 shows the release rates for the different vancomycin loading amounts from the crosslinked ELP constructs at 150 mg/ml. Vancomycin values measured in the supernatant each day were normalized by the starting vancomycin amount and a cumulative release was calculated as a percent of initial. There is evidence of an effect of vancomycin concentration on the release rate from the ELP constructs, with the highest molar loading ratios of compound:ELP associated with the fastest rates of release. The dilute vancomycin concentration maintained in the supernatant drives unsteady solute transport from the ELP construct throughout the duration of the experiment. The crosslinked ELP constructs are able to release vancomycin over at least a 28 day time period.

FIG. 2 demonstrates the vancomycin release rates from the crosslinked ELP constructs with increased concentration of ELP (225 mg/ml). There is evidence of an effect of increased ELP concentration on the release rates from the crosslinked ELP constructs, as compared to the data shown in FIG. 1 for ELP at 150 mg/ml.

### EXAMPLE 6

### Results: Cefazolin Release from the Crosslinked Bioelastomer Constructs

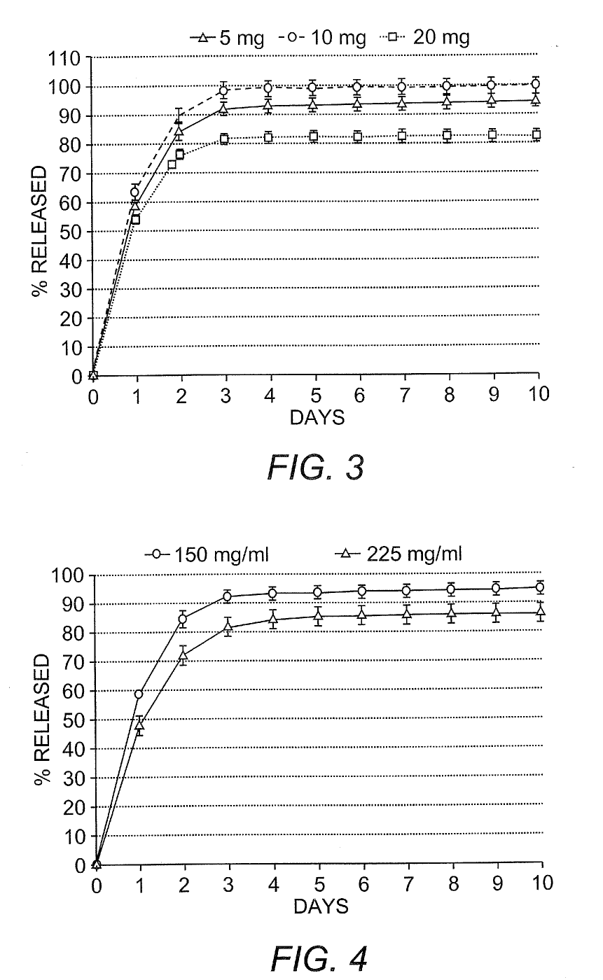

FIG. 3 shows the release rates for the different cefazolin loading amounts from the ELP constructs prepared at an ELP concentration of 150 mg/ml. There is evidence of an effect of cefazolin concentration on the release rate from the ELP constructs, with the highest molar loading ratios of compound:ELP associated with the fastest rates of release. The ELP constructs are able to release vancomycin over at least a 10 day time period, although the majority of cefazolin release took place in the initial two or three days.

FIG. 4 demonstrates the cefazolin release rates from the crosslinked ELP constructs prepared at a higher ELP concentration (225 mg/ml). There is evidence of an effect of increased ELP concentration on the release rate from the ELP construct.

### EXAMPLE 7

### Results: Biological Activity of Vancomycin Released from the Crosslinked Bioelastomer Constructs

There was a zone of bacterial growth inhibition around all supernatants tested including the supernatant of the homogenized final sample. This means that the vancomycin released from the crosslinked ELP constructs, as well as that which remained in the ELP construct, retained biological activity during compound loading and compound release stages.

FIG. 5 is an agar plate showing diameters of zone of inhibition (dark circles) of vancomycin released from a crosslinked bioelastic polymer construct and serial vancomycin dilutions (underlined values). As shown, values for day 1 constructs correspond to ˜1 mg/ml of active compound while day 7 values correspond to less than 0.1 mg/ml active drug. The minimal inhibitory concentration for this antibiotic is reported to be 1.5-3 μg/ml

### EXAMPLE 8

### Results: Biological Activity of Cefazolin Released from the Crosslinked Bioelastomer Constructs

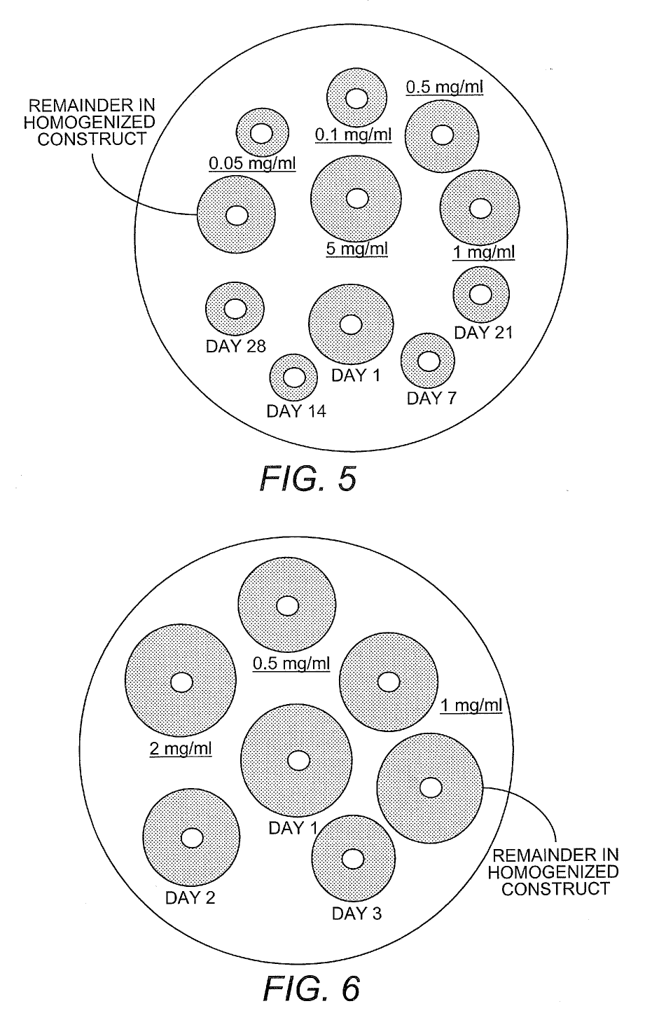

There was a zone of bacterial growth inhibition around days 1-4 and the final homogenized construct for all samples tested. This means that the cefazolin released from the ELP constructs as well as that which remained in the ELP construct was biologically active. FIG. 6 is an agar plate showing diameters of zone of inhibition of cefazolin released from a crosslinked bioelastic polymer construct and serial cefazolin dilutions (underlined values. As shown, values for day 1 constructs correspond to ˜2 mg/ml of active compound while day 3 values correspond to less than 0.5 mg/ml active drug. The minimal inhibitory concentration for this antibiotic is reported to be 0.25-1 μg/ml

### EXAMPLE 9

### Viscosity Testing of Crosslinked Bioelastomer Construct

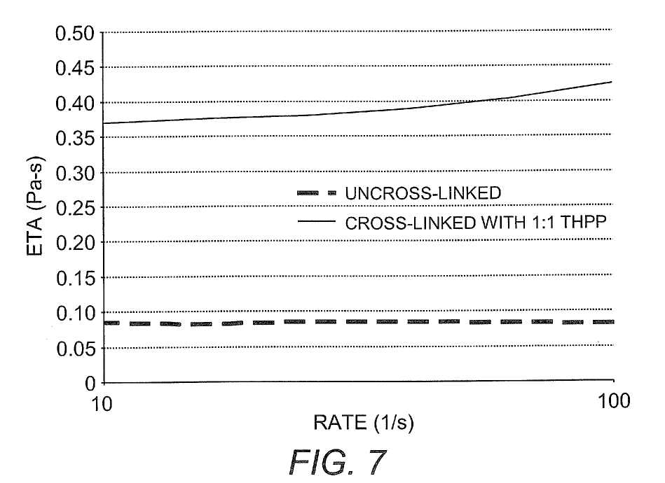

The viscosity of the crosslinked bioelastomer construct was tested at 4° C. in a steady shearing experiment using a cone-on-plate configuration. Viscosity was relatively insensitive to changes in shearing rate. FIG. 7 shows the differences in viscosity of the native bioelastomer and the crosslinked bioelastomer that underwent the steps as described in EXAMPLE 1. The dynamic viscosity of the crosslinked bioelastomer at 10⁻¹ seconds was 0.38 Pa·s. For comparison, the viscosity of honey or pancake syrup is about 2 Pa·s.

The foregoing is illustrative of the present invention, and is not to be construed as limiting thereof. The invention is defined by the following claims, with equivalents of the claims to be included therein.

That which is claimed is:

1. A method for delivering a compound of interest to a selected region in a subject, said method comprising:
administering a composition to said region of interest, said composition comprising a cross-linked bioelastomer in combination with said compound of interest non-covalently combined with said cross-linked bioelastomer.

2. The method of claim 1, said composition produced by the process of cross-linking a bioelastomer to produce a cross-linked polymer thereof, and then combining said cross-linked polymer with said compound of interest.

3. The method of claim 1, said composition produced by the process of cross-linking a bioelastomer to produce a cross-linked polymer thereof, drying said cross-linked polymer, rehydrating said cross-linked polymer in the presence of a quenching agent in an aqueous solution in an amount sufficient to inactivate remaining reactive sites on said cross-linked polymer and again drying said cross-linked and quenched polymer, then combining said cross-linked and quenched polymer with said compound of interest, in solution, to produce said composition with said compound of interest non-covalently combined with said cross-linked polymer.

4. The method of claim 1, said composition comprising said compound of interest and said cross-linked polymer at a molar ratio of at least 0.5 to 1, compound to polymer.

5. The method of claim 1, wherein said compound of interest is a protein or peptide, said composition comprising said compound of interest and said cross-linked polymer at a molar ratio of at least 0.05 to 1.

6. The method of claim 2, wherein said cross-linking agent is THPP and said quenching agent is glycine.

7. The method of claim 1, wherein said administering step is carried out by injection.

8. The method of claim 1, wherein said region of interest is a joint.

9. The method of claim 1, wherein said region of interest is an intervertebral disc space.

10. The method of claim 1, wherein said administering step is carried out by intra-articular injection.

11. The method of claim 1, wherein said patient is afflicted with diarthrodial joint disorder and/or intervertebral disc pathology, or infection.

12. The method of claim 1, wherein said compound of interest is a protein or peptide.

13. The method of claim 1, wherein said compound of interest is an antibody.

14. The method of claim 1, wherein said compound of interest is an antibiotic.

15. The method of claim 1, wherein said compound of interest is an antifungal.

16. The method of claim 1, wherein said region of interest is a joint, said compound of interest is an antiinflammatory compound, said patient is afflicted with osteoarthritis, and said composition is administered in an amount effective to treat said osteoarthritis.

17. A method of making a composition useful for delivering a compound of interest to a selected region in a subject, said method comprising:
cross-linking a bioelastomer to produce a cross-linked polymer thereof, and then
combining said cross-linked bioelastomer with said compound of interest to produce said composition, with said compound of interest non-covalently combined with said cross-linked polymer.

18. The method of claim 17, further comprising the steps of drying said cross-linked polymer and rehydrating said cross-linked polymer prior to said combining step.

19. The method of claim 17, further comprising the steps of drying said cross-linked polymer and rehydrating said cross-linked polymer prior to said combining step in an aqueous solution of volume not greater than the volume of water lost therefrom during said drying step.

20. The method of claim 17, further comprising the steps of quenching said cross-linked polymer prior to said combining step with a quenching agent in an amount sufficient to inactivate remaining active sites on said cross-linked polymer.

21. The method of claim 17, further comprising the steps of quenching said cross-linked polymer prior to said combining step with a quenching agent in an amount sufficient to inactivate remaining reactive sites on said cross-linked polymer and in an aqueous solution of volume not greater than the volume of water lost therefrom during said drying step.

22. A kit useful for making a composition for delivering a compound of interest to a selected region in a subject, said kit comprising:
a first reagent, said first reagent produced by the process of cross-linking an elastin-like peptide to produce a cross-linked polymer thereof, and
a second reagent comprising said compound of interest non-covalently entrapped in said cross-linked polymer.

23. The kit of claim 22, wherein said cross-linked polymer is quenched to inactivate remaining reactive sites thereon.

24. The method of claim 1 where the resulting crosslinked bioelastomer exhibits a phase transitioning behavior, as does the uncrosslinked native bioelastomer.

25. The method of claim 1 where the phase transitioning behavior of said crosslinked bioelastomer is reversible and repeatable.

26. The method of claim 1 where the composition has a viscosity suitable for injection or delivery through a syringe.


SEQUENCE LISTING (14 sequences)

SEQ ID NO: 1 - protein, 5 aa, Artificial
  Bioelastic polymer repeat unit
VPGXG

SEQ ID NO: 2 - protein, 40 aa, Artificial
  Bioelastic polymer repeat unit
VPGAGVPGAGVPGAGVPGAGVPGAGVPGVGVPGGGVPGGG

SEQ ID NO: 3 - protein, 4 aa, Artificial
  Bioelastic polymer repeat unit
VPGG

SEQ ID NO: 4 - protein, 4 aa, Artificial
  Bioelastic polymer repeat unit
GGVP

SEQ ID NO: 5 - protein, 4 aa, Artificial
  Bioelastic polymer repeat unit
GGFP

SEQ ID NO: 6 - protein, 4 aa, Artificial
  Bioelastic polymer repeat unit
GGAP

SEQ ID NO: 7 - protein, 5 aa, Artificial
  Bioelastic polymer repeat unit
VPGVP

SEQ ID NO: 8 - protein, 5 aa, Artificial
  Bioelastic polymer repeat unit
GVGVP

SEQ ID NO: 9 - protein, 5 aa, Artificial
  Bioelastic polymer repeat unit
GKGVP

SEQ ID NO: 10 - protein, 5 aa, Artificial
  Bioelastic polymer repeat unit
GVGFP

SEQ ID NO: 11 - protein, 5 aa, Artificial
  Bioelastic polymer repeat unit
GFGFP

SEQ ID NO: 12 - protein, 5 aa, Artificial
  Bioelastic polymer repeat unit
GEGVP

SEQ ID NO: 13 - protein, 5 aa, Artificial
  Bioelastic polymer repeat unit
GFGVP

SEQ ID NO: 14 - protein, 5 aa, Artificial
  Bioelastic polymer repeat unit
GVGIP